SEQUENCE LISTING (19 sequences)

SEQ ID NO: 1 - DNA, 528 bp, Arabidopsis thaliana
  source 1..528: mol_type = genomic DNA
atgtctataaatataagagatcctcttatagtaagcagagttgttggagacgttcttgatccgtttaatagatcaatcactctaaaggttacttatagccaaagagaggtgactaatggcttggatctaaggccttctcaggttcaaaacaagccaagagttgagattggtggagatgacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagtagactttag

SEQ ID NO: 2 - protein, 768 aa, unidentified
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSSRADPKKKRKV

SEQ ID NO: 3 - protein, 1379 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 4 - protein, 187 aa, Streptococcus pyogenes
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 5 - DNA, 20 bp, synthetic construct
  source 1..20: mol_type = other DNA
ttggtagtagcgactccatg

SEQ ID NO: 6 - DNA, 20 bp, synthetic construct
  source 1..20: mol_type = other DNA
tacagttaactatttggagg

SEQ ID NO: 7 - DNA, 20 bp, synthetic construct
  source 1..20: mol_type = other DNA
ctcttgccagcaatgcttgg

SEQ ID NO: 8 - DNA, 20 bp, synthetic construct
  source 1..20: mol_type = other DNA
gattgccctccaagcattgc

SEQ ID NO: 9 - DNA, 20 bp, synthetic construct
  source 1..20: mol_type = other DNA
gtagtagcgactccatgggg

SEQ ID NO: 10 - DNA, 20 bp, synthetic construct
  source 1..20: mol_type = other DNA
actatttggaggcggcgtta

SEQ ID NO: 11 - DNA, 20 bp, synthetic construct
  source 1..20: mol_type = other DNA
gttgggcgtgaggaagtacg

SEQ ID NO: 12 - DNA, 20 bp, synthetic construct
  source 1..20: mol_type = other DNA
cctccagcaatatcggtttg

SEQ ID NO: 13 - DNA, 107 bp, unidentified
  source 1..107: mol_type = other DNA
aggtagtctncattgattatccaagaccggaggtaggcagnncagttatatattgaagctcaagacggtttaaagtgttaaggactggatgagaaagcaagtatgtg

SEQ ID NO: 14 - DNA, 100 bp, unidentified
  source 1..100: mol_type = other DNA
gtgagcgagagagccanagatagaagcacgaagggtacnatcgtgccgtatgcttggagggcaatcttatgttgaagctcaagacggtttaagtgttaag

SEQ ID NO: 15 - DNA, 116 bp, unidentified
  source 1..116: mol_type = other DNA
tgggaactgaaagtcaagatggtcgcttgcaaaggaatttgttatgttttggtagtagcgactccttggagggcaatcttatgttgaagctcaagacggtttaagtgttaaggact

SEQ ID NO: 16 - DNA, 100 bp, unidentified
  source 1..100: mol_type = other DNA
gcattgattatccaagaccggaggtagacaatacagttaattatttggttggagggcaatcttatgttgaagctcaagacggtttaagtgttaaggactg

SEQ ID NO: 17 - DNA, 93 bp, unidentified
  source 1..93: mol_type = other DNA
gatcttgatgaatcaagatcaagacttggagaaagacttagaattcatgcttctgctgatgatcttagagctttgcttgctagaccttggctt

SEQ ID NO: 18 - DNA, 93 bp, unidentified; identical to SEQ ID NO: 17
  source 1..93: mol_type = other DNA

SEQ ID NO: 19 - DNA, 94 bp, unidentified
  source 1..94: mol_type = other DNA
tttcgttgagcagcacaagcactacttggatgagatcatcgagcagatctctgagttctctaaaagggtgatcctcgctgatgcaaacctcgaa

(19) United States
(12) Patent Application Publication
(10) Pub. No.: US 2025/0327086 A1
(43) Pub. Date: Oct. 23, 2025

(54) TISSUE-CULTURE INDEPENDENT GENE EDITING OF CELLS BY A LONG-DISTANCE RNA TRANSPORT SYSTEM

(71) Applicant: Texas Tech University System, Lubbock, TX (US)

(72) Inventors: Benjamin Perez-Sanchez, Lubbock, TX, TX (US); Damar L. Lopez-Arredondo, Lubbock, TX, TX (US); Luis R. Herrera-Estrella, Lubbock, TX, TX (US)

(73) Assignee: Texas Tech University System, Lubbock, TX (US)

(21) Appl. No.: 18/290,113

(22) PCT Filed: Apr. 20, 2022
(86) PCT No.: PCT/US2022/025621

§ 371 Date: Nov. 9, 2023

Related U.S. Application Data
(60) Provisional application No. 63/177,033, filed on Apr. 20, 2021.

(51) Int. Cl. C12N 15/82; C12N 9/22; C12N 15/11

(52) U.S. Cl. CPC C12N 15/8213; C12N 9/226; C12N 15/111; C12N 2310/20

(57) ABSTRACT

In an embodiment, the present disclosure relates to a method of editing at least one gene in plant target cells. The method generally includes introducing genetic components of a gene editing system to a first region of the plant. The genetic components are then transported from the first region to the second region, which is different from the first region. The genetic components are processed in the cells in the second region to form the gene editing system such that the gene editing system edits the at least one gene in the cells. The gene edited cells give rise to gametes that produce gene edited seeds upon fertilization.

### CROSS-REFERENCE TO RELATED APPLICATIONS

The present application claims the benefit of U.S. Provisional Patent Application No. 63/177,033, filed on Apr. 20, 2021. The entirety of the aforementioned application is incorporated herein by reference.

### STATEMENT REGARDING FEDERALLY SPONSORED RESEARCH

This invention was made with government support under 2021-67013-34738 awarded by the U.S. Department of Agriculture. The government has certain rights in the invention

Pursuant to 37 C.F.R. § 1.834, Applicant has submitted a sequence listing in XML format (“Sequence Listing”). The name of the file containing the Sequence Listing is “AF13368.P022WOUS.XML”. The date of the creation of the Sequence Listing is May 18, 2024. The size of the Sequence Listing is 21,000 bytes. Applicant hereby incorporates by reference the material in the Sequence Listing.

### BACKGROUND

Several tissue culture-independent transformation and gene editing protocols have been developed for use in plants. However, there are still several challenges that need to be solved to create a facile, species-independent, efficient, and reproducible system to produce gene-edited plants. Various embodiments of the present disclosure seek to address the aforementioned challenges.

### SUMMARY

In an embodiment, the present disclosure relates to a method of editing at least one gene in plant target cells. The method generally includes introducing genetic components of a gene editing system to a first region of the plant. Thereafter, the genetic components are transported from the first region of a plant to a second region of the plant, which contains the target cells. Next, the genetic components are processed in the target cells in the second region to form the gene editing system. The gene editing system then edits at least one gene in the cells. In some embodiments (e.g., embodiments where the cells are gamete-producing cells), gene edited gametes can then be formed and, upon fertilization, form a zygote that will form gene edited seeds.

### DESCRIPTION OF THE DRAWINGS

FIG. 1 depicts a method of editing at least one gene in plant target cells according to an aspect of the present disclosure.

FIGS. 2A and 2B provide schematic representations of the N. benthamiana PDS2 gene. FIG. 2A illustrates Cas9 target regions. FIG. 2B illustrates Cas Φ target regions. Brackets indicate the distance between the edges of the guide RNAs.

FIGS. 3A and 3B provide schematic representations of the pREP_L2cc binary plasmid and Replicon formation. FIG. 3A shows an empty Golden Gate Level 2 pREP_L2acc vector. The vector contains the E. coli lacZα flanked by BsaI Type IIS restriction enzyme sites. The genetic components flanked by the Left and Right borders (LB and RB) can be integrated into plant cells by A. tumefaciens and R. rhizogenes strains. FIG. 3B illustrates the formation of the empty replicon after the BeYDV Rep gene expression in plant cells. The genetic components flanked by the LIR region from the viral replicon can be contained within the vector.

FIG. 4 provides a schematic of the constructions assembled within the pREP_L2acc vector.

FIG. 5 provides initial Fast-TrACC protocol testing the transgene induction of A. tumefaciens GV3101 harboring p1R. The fluorescence signal was highly variable between seedlings, with just a few plants showing high fluorescence.

FIG. 6 illustrates that R. rhizogenes 18r12 is more efficient in inducing transgene expression under the modified Fast-TrACC protocol.

FIG. 7 shows fluorescence signals in plant seedlings, indicating the expression of gene editing reagents summarized in Example 1. The plants shown were pooled, and total DNA was extracted for PCR amplification of the PDS genes.

FIG. 8 shows provides DNA sequences, where seedlings infiltrated with editing reagents are represented by aberrant base calls near the Cas9 cut sites. Electropherograms of the pooled sequenced PDS2 gene show that regions close to the expected cut sites show overlapped signals coming from different nucleotides simultaneously. The sequences are also provided in SEQ ID NO: 13 and SEQ ID NO: 14.

FIG. 9 illustrates that the Vector combination and Single Transcript Unit approach can induce targeted mutations. The upper panel shows PDS2 gene deletion found in the pooled DNA from seedlings treated with the p7sturR vector. The lower panel shows PDS2 gene deletion was found in the plants treated with p6CFRP and p7GFRP. The sequences are also provided in SEQ ID NO: 15 and SEQ ID NO: 16.

FIG. 10 provides a basic schematic representation of an in-planta gene-editing strategy.

FIG. 11 shows six-week-old N. benthamiana abaxial surface infiltrated with A. tumefaciens. Four infiltrations were made on each leave.

FIG. 12 provides RT-PCR electrophoresis of RNA extracted from agro-infiltrated N. benthamiana plants. Here, the presence of the Cas9, Csy4, and three reference gene transcripts in the apexes of six plants was evaluated. Apexes m1, m2, and m3 correspond to the plants infiltrated with p6CFRP and p7GFRP. Apexes c1, c2, and c3 correspond to plants infiltrated with the empty pREP_L2acc. Total extracted RNA from m3 was used as a negative RT PCR control (--). The genes used as reference were NbNQO Niben101Scf00578g05006.1 NAD (P) H dehydrogenase (quinone) and NbUBE35 Niben101Scf00398g00015.1 ubiquitin-conjugating enzyme 35. Reference gene NbUBE35 was PCR amplified again at a lower alignment temperature.

FIG. 13 provides sanger sequencing, which reveals that the amplified transcripts correspond to Csy4 and Cas9. Electropherograms show the sequence of the amplified RT products using primers corresponding to Csy4 and Cas9 in the m1 and m3 apexes. The sequences are also provided in SEQ ID NO: 17, SEQ ID NO: 18, an SEQ ID NO: 19.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Genomic information is rapidly being generated not only for major crops such as maize, wheat and rice, but also for other crops including common beans, chili peppers, papaya, grapevines, and the like. The increasing amount of information about plant genomes has allowed the use of different strategies, such as genome-wide association studies, comparative genomics, and use of mutant and mapping populations to identify genes that contribute to different traits important for plant productivity.

Gene transfer by Agrobacterium tumefaciens-based protocols are commonly used in many crops for gene identification purposes. However, the effectiveness and efficiency of such techniques are very much species- and genotype-dependent, and in most cases rely upon time consuming and cumbersome tissue culture-based protocols.

The clustered regularly interspaced short palindromic repeats (CRISPR)/Cas nuclease (Cas) system (CRISPR/Cas system), such as the CRISPR/Cas 9 system, can be applied to any plant species, provided that an efficient system to produce or introduce the necessary proteins and RNAs for gene editing is available. However, the complexity of plant regeneration has become a major hurdle for the general application of the CRISPR/Cas system on many plant species.

A report describing a method for the introduction of genetic material into pollen cells has been published. In this method, denominated “magnetofection”, magnetic nanoparticles are used to introduce DNA into pollen cells. These methods rely on the presence of pores in the cell wall of pollen cells that allow the entry of nanoparticles to deliver DNA. Transformation efficiency using this method is about 0.5% of the seed collected from flowers fertilized with magnetofected pollen. This method reduces the time to get transgenic seeds to less than 6 months and was reported to work with different constructs. The same procedure could be used to introduce or transiently express Cas9 and guide RNAs for gene editing in pollen. However, to identify genome edited events using this technology when the phenotype cannot be selected is far more challenging.

More recently, a system of genome editing by de novo induction of meristems was reported. This system is based on the use of developmental regulators (DRs) whose ectopic expression was previously shown to induce the de novo formation of somatic embryos or shoot meristems. However, a significant number of the generated shoots showed phenotypical abnormalities. Although in principle this method should be applicable to all plant species, a single combination of DRs is unlikely to work for all plant species. Therefore, the combination of DRs and their expression level needs to be optimized for each plant species.

Moreover, the de novo meristem formation system must combine optimized expression of the plant morphogenic genes with a robust method to eliminate them or limit their expression after plant transformation or regeneration has occurred and their function is no longer required. Different species and even different varieties within the same species require new combinations of morphogenic triggers (new combinations of genes or varied expression patterns) to produce new apical meristems for rapid production of genetically modified or gene edited plants.

Although promising strategies for genome editing have been developed, there is still a need for more facile, reproducible, and versatile methods to facilitate the application of gene editing strategies for plant species for which transformation or regeneration procedures are difficult or have not been developed yet. In sum, a need exists for more effective gene editing systems and methods for a large variety of plants. Various embodiments of the present disclosure address the aforementioned need.

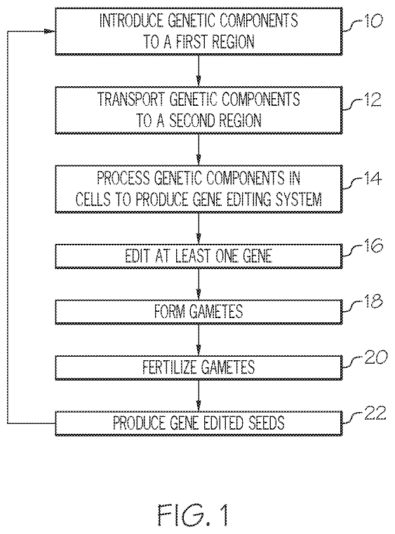

In some embodiments, the present disclosure pertains to methods of editing at least one gene in plant target cells. In some embodiments illustrated in FIG. 1, the methods of the present disclosure can include one or more of the following steps: introducing genetic components of a gene editing system to a first region of the plant (step 10); transporting the genetic components (including their transcripts) from the first region of the plant to a second region of the plant having target cells (step 12); processing the genetic components in the target cells in the second region to form the gene editing system (step 14); editing at least one gene in the target cells (step 16); forming gametes from the target cells (step 18); fertilizing the formed gametes (step 20); and producing gene edited seeds (step 22). In some embodiments, the methods of the present disclosure can be repeated multiple times.

As set forth in more detail herein, the gene editing methods of the present disclosure can be utilized in various plants and/or plant varieties, plant regions, and on various target cells. Additionally, the gene editing methods of the present disclosure can utilize various gene editing systems. As also set forth in more detail herein, the gene editing systems can have numerous genetic components, modes of introduction, and transport means. Moreover, the genetic components of the gene editing systems of the present disclosure can be processed in various manners and edit various genes through different modes, thereby causing varying effects. The gene editing methods of the present disclosure can also have numerous advantages.

### Plants

The gene editing methods of the present disclosure can be applied to various plants. For instance, in some embodiments, the plants can include, without limitation, maize, rice, soybean, cotton, wheat, N. benthamiana, Arabidopsis, tobacco, tomato, lettuce, common beans, potato, grapes, varieties thereof, and combinations thereof. In some embodiments, the plant is soybean. In some embodiments, the plant is tobacco. In some embodiments, the plant is N. benthamiana. In some embodiments, the plant is a Solanaceae.

### Plant Regions

As described in further detail herein, the plants of the present disclosure can have various regions. For instance, in some embodiments, the plants have a first region and a second region. In some embodiments, the first region is different from the second region. For example, in some embodiments, the first region includes, without limitation, leaves, stems, cotyledons, and combinations thereof. In some embodiments, the second region includes, without limitation, shoot apical meristems (SAM), floral meristems, inflorescence meristems, root apical meristem, lateral meristems, and combinations thereof. In some embodiments, the second region includes a root apical meristem. In some embodiments, the second region includes a shoot apical meristem. In some embodiments, the second region includes a floral meristem.

In some embodiments, each of the first region and the second region includes a single location of the plant. In some embodiments, each of the first region and the second region includes a plurality of different locations of the plant. For instance, in some embodiments, the first region includes two different locations of the plant. In more specific embodiments, the two different locations include two different leaves of the plant.

### Target Cells

The target cells that are modified by the gene editing methods of the present disclosure can also have numerous embodiments. For instance, in some embodiments, the target cells are gamete-generating cells (i.e., cells that are capable of forming gametes or give rise to gamete forming cells). In some embodiments, the gametes are capable of forming or can form seeds upon fertilization. In some embodiments, the target cells are meristematic cells, such as, for example, shoot apical meristematic cells, floral meristematic cells, inflorescence meristem cells, root apical meristematic cells, lateral meristem cells, and combinations thereof. In some embodiments, the target cells include shoot apical meristematic cells. In some embodiments, the target cells include floral meristematic cells.

### Genetic Components

Genetic components generally refer to components that can be processed, transcribed, and/or translated to form the gene editing systems of the present disclosure. As described in further detail herein, the gene editing methods of the present disclosure can introduce various genetic components to first regions of plants. For instance, in some embodiments, the genetic components are introduced in the form of RNA, DNA, proteins, or combinations thereof.

In some embodiments, the genetic components are introduced in the form of RNA. Thereafter, the RNA is transported to the second region of the plant.

In some embodiments, the genetic components are introduced in the form of RNA and proteins (e.g., Ribonucleoproteins). Thereafter, the RNA and proteins are transported to the second region of the plant. In some embodiments, the proteins are Cas 9 and Csy 4 linked to a protein domain that allows long distance transport and entry to the second region (e.g., the root apical meristem and/or shoot apical meristem). In some embodiments, the RNA is comprised of guide RNAs (i.e., sgRNAs) and a transport signal for an RNA transport system of the plant.

In some embodiments, the genetic component is introduced in the form of DNA, which in turn is transcribed to RNA (e.g., mRNA) in the first region and then transported to the second region. In some embodiments, the DNA of the genetic components are contained in one or more expression vectors.

In some embodiments, the RNA includes transcripts of the gene editing system. In some embodiments, the genetic component is RNA that includes a transport signal for an RNA transport system of the plant. In some embodiments, the transport signal is operative for facilitating the transport of the RNA from the first region to the second region of the plant by the RNA transport system. In some embodiments, the transport signal includes a sequence from the Flowering Locus T gene. In some embodiments, the DNA sequence of the Flowering Locus T gene includes SEQ ID NO: 1, or a sequence that shares at least 65% sequence identity to SEQ ID NO: 1. In some embodiments, the transport signal includes, without limitation, FLOWERING LOCUS T (FT) 5′ untranslated region (UTR), complete or partial FLOWERING LOCUS T transcribed region, GA-INSENSITIVE (GAI) UTRs, CENTRORADIALIS UTRs, tRNA-like elements, and combinations thereof. In some embodiments, the RNA transport signal mobilizes Csy 4 mRNA, and/or Cas 9 mRNA or CasΦ mRNA alone or linked to the sgRNAs to the shoot apical meristem after agroinfiltration of leaves.

In some embodiments, the introduced RNA encodes Cas 9 and sgRNAs linked to a transport signal for an RNA transport system of the plant. In some embodiments, the genetic component is the genetic component for the Cas 9/sgRNA system. In some embodiments, the genetic component also includes the genetic component of the Csy 4 endonuclease, an endonuclease that processes the Cas9/sgRNA.

In some embodiments, the genetic component is a DNA construct expressing a Cas 9/sgRNA polycistronic mRNA linked to an RNA transport signal using a constitutive promoter in a vector carrying a geminiviral origin of replication and a Csy 4 construct using a SAM-specific promoter. In some embodiments, the aforementioned genetic components are utilized such that guide RNA processing only takes place in meristematic cells and not in cotyledon cells (i.e., where most of the Cas 9/sgRNA RNA is produced). In some embodiments, the Cas 9/sgRNA mRNA is linked to an RNA motif to mobilize RNA to the SAM cells. In some embodiments, Cas 9 nuclease and the mature RNA guides are produced in SAM cells to achieve gene editing. In some embodiments, the Csy 4 mRNA linked to RNA transport signal mobilizes and enters the same cells as the Cas 9/sgRNA polycistronic mRNA, where it is processed to produce all the necessary elements for gene editing. In some embodiments, processing of the Cas 9/sgRNA polycistronic mRNA is processed into independent components by remains of ribozymes inserted in between each element.

### Modes of Introduction of the Genetic Components

Various methods may be utilized to introduce genetic components to the first regions of plants. For instance, in some embodiments, the mode of introduction includes, without limitation, transfection, electroporation, particle bombardment, agrofiltration, and combinations thereof. In some embodiments, the mode of introduction is agroinfiltration.

In some embodiments, the mode of introduction is conducted through a bacterial host strain carrying the genetic components. In some embodiments, the bacterial host strain is A. tumefaciens. In some embodiments, the bacterial host strain is R. rhizogenes. In some embodiments, the bacterial host strain introduces the genetic components to the first region, where the genetic components are transiently expressed.

The genetic components of the present disclosure can be introduced into various locations within a first region of a plant. For instance, in some embodiments, the genetic components are introduced at the same location of the first region of the plant. In some embodiments, the genetic components are introduced at different locations of the first region of the plant.

In some embodiments, the genetic components include a plurality of different components. In some embodiments, the different components within the plurality of components are introduced at the same location of the first region. In some embodiments, the different components are introduced at different locations of the first region. In some embodiments, the different components are introduced at two different locations of the first region. In some embodiments, the different components include two or more different leaves of the plant.

In some embodiments, the genetic components are introduced at two different locations of the first region of the plant. For instance, in some embodiments, genetic components for the Cas 9/sgRNA system are introduced into a first location of the first region of the plant while the genetic components for the Csy 4 endonuclease is introduced to the second location of the first region of the plant.

Transport from First Region to Second Region

The genetic components of the present disclosure can be transported from the first region to the second region of plants through various linkages within the plants. For example, in some embodiments, the transport occurs through the phloem of the plant. In some embodiments, the linkage facilitates the transportation of the genetic component from the first region of the plant to the second region of the plant.

In some embodiments, transportation of the genetic components from the first region to the second region of the plants can occur through an RNA transport system. In some embodiments, the RNA transport system is a long-distance RNA transport system. In some embodiments, the RNA transport system is an mRNA-binding protein-mediated transport system. In some embodiments, the RNA transport system is a flowering locus T (FT) protein 1 RNA mobility system

In some embodiments, the RNA transport system includes transfer RNAs (tRNAs) that are known to be transported through a plant's vascular system. For instance, a recent study showed that mRNAs harboring specific tRNA structures in its 3′UTR move from transgenic roots of composite plants into wild-type leaves and from transgenic leaves into wild-type flowers and roots. See, e.g., Plant Cell, 2016, 28:1237-1249.

After transportation from the first region of the plant to the second region of the plant, the genetic components of the present disclosure can be processed by target cells by various methods. For instance, in some embodiments, the genetic components are processed through translation. In some embodiments, the genetic components are processed through enzymatic digestion (e.g., endonuclease digestion, such as by the Csy 4 endonuclease). In some embodiments, the genetic components are processed by autocatalytic ribozymes. In some embodiments, the genetic components are processed through RNA processing.

### Gene Editing

The methods of the present disclosure may be utilized to edit various genes. For instance, in some embodiments, the genes to be edited include endogenous genes in the plant target cells. In some embodiments, the genes to be edited include exogenous genes that are to be inserted into the plant target cells.

The methods of the present disclosure can be utilized to edit genes in various manners. For instance, in some embodiments, gene editing refers to introducing a mutation to the gene, introducing a deletion to the gene, introducing an insertion to the gene, removing a portion of the gene, changing a base of the gene, removing the gene, inserting the gene, partially or fully replacing the gene, and combinations thereof. In some embodiments, gene editing may include inserting a new gene into a specific genome location in the plant target cells.

### Gene Editing Systems

The gene editing systems of the present disclosure can have numerous embodiments. Moreover, the gene editing systems of the present disclosure can edit various genes via numerous modes, thereby resulting in various effects.

In specific embodiments, the gene editing systems of the present disclosure include a clustered regularly interspaced short palindromic repeats (CRISPR)/Cas nuclease (Cas) system (CRISPR/Cas system). In some embodiments, the CRISPR/Cas system includes at least one Cas nuclease, and at least one guide RNA.

In some embodiments, the genetic components of the CRISPR/Cas system include the genetic component of the Cas nuclease, and a guide RNA precursor. In some embodiments, the genetic components of the CRISPR/Cas system further include the genetic component of a guide RNA nuclease. In some embodiments, the guide RNA nuclease is operable to convert the guide RNA precursor to the guide RNA.

In some embodiments, the genetic components of the CRISPR/Cas system further include at least one transport sequence. In some embodiments, the at least one transport sequence is recognizable by an RNA transport system for facilitating the transport of the genetic components from a first region of a plant to a second region of the plant.

Cas nucleases generally refer to RNA-guided DNA endonuclease enzymes. The CRISPR/Cas systems of the present disclosure can utilize numerous Cas nucleases. For instance, in some embodiments, the Cas nuclease includes, without limitation, class 2 of Cas nucleases, Cas 9, Cas Φ, CasΦ2, Cpf1, or combinations thereof. In some embodiments, the Cas nuclease is fused to at least one nuclear localization peptide.

In some embodiments, the Cas nuclease includes CasΦ. In some embodiments, CasΦ also includes a nuclear localization peptide (e.g., an SV40 nuclear localization peptide) for directing CasΦ to the nucleus of cells. In some embodiments, the protein sequence of CasΦ includes SEQ ID NO: 2, or a sequence that shares at least 65% sequence identity to SEQ ID NO: 2.

In some embodiments, the Cas nuclease includes Cas 9. In some embodiments, Cas 9 also includes a nuclear localization peptide (e.g., an SV40 nuclear localization peptide) for directing Cas 9 to the nucleus of cells. In some embodiments, the protein sequence of Cas 9 includes SEQ ID NO: 3, or a sequence that shares at least 65% sequence identity to SEQ ID NO: 3.

Guide RNAs generally refer to RNA sequences that can guides Cas nucleases to a particular DNA sequence. The CRISPR/Cas systems of the present disclosure can also utilize numerous guide RNA nucleases. In some embodiments, the guide RNA nuclease includes Csy 4. In some embodiments, the protein sequence of Csy 4 includes SEQ ID NO: 4, or a sequence that shares at least 65% sequence identity to SEQ ID NO: 4.

In some embodiments, the genetic components of the CRISPR/Cas system are introduced to first regions of plants in the form of DNA, RNA, Ribonucleoproteins, or combinations thereof. In some embodiments, the genetic components of the CRISPR/Cas system are introduced in the form of DNA. In some embodiments, the DNA encodes at least one guide RNA and at least one Cas nuclease. In some embodiments, the DNA is transcribed into one or more RNAs in a first region of a plant. Thereafter, the one or more RNAs are transported to a second region of the plant and processed in the target cells of the plant in the second region to form the CRISPR/Cas system. In some embodiments, the processing includes translation of the one or more RNAs to form the Cas nuclease, the cutting of the one or more RNAs by the Cas nuclease to form the guide RNA, and the association of the Cas nuclease with the formed guide RNA to form the CRISPR/Cas system.

In some embodiments, the DNA of the genetic components are contained in a single expression vector. In some embodiments, the DNA of the genetic components are contained in a first and a second expression vector. In some embodiments, the first expression vector expresses the Cas nuclease, and the second expression vector expresses the guide RNA. In some embodiments, the Cas nuclease includes CasΦ. In some embodiments, CasΦ also includes a nuclear localization peptide (e.g., an SV40 nuclear localization peptide) for directing CasΦ to the nucleus of cells.

In some embodiments, the DNA of the genetic components of the CRISPR/Cas system encodes at least one guide RNA, at least one Cas nuclease, and at least one guide RNA nuclease. In some embodiments, the DNA is transcribed into one or more RNAs in a first region of a plant. Thereafter, the one or more RNAs are transported to a second region of the plant and processed in the target cells of the plant in the second region to form the CRISPR/Cas system. In some embodiments, the processing includes translation of the one or more RNAs to form the Cas nuclease and the guide RNA nuclease, the cutting of the one or more RNAs by the guide RNA nuclease to form the guide RNA, and the association of the Cas nuclease with the formed guide RNA to form the CRISPR/Cas system.

In some embodiments, the DNA of the aforementioned genetic components are contained in a single expression vector. In some embodiments, the DNA of the aforementioned genetic components are contained in a first and a second expression vector. In some embodiments, the first expression vector expresses the Cas nuclease and the guide RNA and the second expression vector expresses the guide RNA nuclease.

In some embodiments, the Cas nuclease includes Cas 9. In some embodiments, Cas 9 also includes a nuclear localization peptide (e.g., an SV40 nuclear localization peptide) for directing Cas 9 to the nucleus of cells.

In some embodiments, the guide RNA nuclease includes Csy 4. In some embodiments, the protein sequence of Csy 4 includes SEQ ID NO: 3, or a sequence that shares at least 65% sequence identity to SEQ ID NO: 3.

In some embodiments, the CRISPR/Cas genetic components can also include at least one transport sequence that is recognizable by an RNA transport system for facilitating the transport of the genetic components from the first region to the second region. In some embodiments, the transport sequence includes a sequence from the Flowering Locus T gene.

As set forth herein, the genetic components of the CRISPR/Cas systems of the present disclosure can be introduced to a first region of a plant in the form of DNA. The DNA can then be transcribed into RNA and then transported to a second region of the plant.

In alternative embodiments, the genetic components of the CRISPR/Cas system are introduced in the form of one or more RNAs, which are then directly transported to the second region of the plant. In some embodiments, the one or more RNAs can be introduced onto the first region of the plant by particle bombardment, electroporation or combined with nanotubes to enter the cells.

In some embodiments, the one or more RNAs include a precursor to the at least one guide RNA, and a messenger RNA for the at least one Cas nuclease. In some embodiments, the one or more RNAs are transported to the second region, where they are processed in the target cells to form the CRISPR/Cas system. In some embodiments, the processing includes translation of the messenger RNA to form the Cas nuclease, the cutting of the precursor to the at least on guide RNA by the Cas nuclease to form the guide RNA, and the association of the Cas nuclease with the formed guide RNA to form the CRISPR/Cas system.

In some embodiments, the one or more RNAs include a precursor to the at least one guide RNA, a messenger RNA for the at least one Cas nuclease, and a messenger RNA for at least one guide RNA nuclease. In some embodiments, the one or more RNAs are transported to the second region, wherein they are processed in the target cells in the second region to form the CRISPR/Cas system. In some embodiments, the processing includes translation of the Cas messenger RNAs to form the Cas nuclease, the translation of the guide RNA nuclease messenger RNA to form the guide RNA nuclease, the cutting of the precursor to the at least on guide RNA by the guide RNA nuclease to form the guide RNA, and the association of the Cas nuclease with the formed guide RNA to form the CRISPR/Cas system.

In some embodiments, the one or more RNAs also include at least one transport sequence. In some embodiments, the at least one transport sequence is recognizable by an RNA transport system for facilitating the transport of the one or more RNAs from the first region to the second region.

In more specific embodiments, the gene editing systems of the present disclosure include Cas 9/single-guide RNA (sgRNA) and Csy4 endonuclease. In some embodiments, the Csy 4 endonuclease processes the Cas 9/sgRNA in the target cells in order to activate the gene editing system. The use of additional gene editing systems can also be envisioned. In some embodiments, the gene editing system includes a nuclease. In some embodiments, the nuclease is a member of the class 2 of Cas nucleases. In some embodiments, the nuclease is Cas9, Cpf1, CasΦ2 or any other DNA nuclease that can be targeted to specific DNA sequences via sgRNAs.

### Targeted Genes

The gene editing systems of the present disclosure can be utilized to edit one or more of various genes in target cells. For instance, in some embodiments, the gene editing systems of the present disclosure can be utilized to edit a phytoene desaturase (PDS) gene. In some embodiments, the gene editing system can edit one or more PDS gene orthologs. In some embodiments, the gene editing systems can edit two PDS gene orthologs. In some embodiments, the two PDS gene orthologs are simultaneously targeted. In some embodiments, the gene editing systems of the present disclosure can edit and/or target magnesium chelatase subunit I (CHLI). In some embodiments, the gene editing system can modify one or both of PDS and CHLI. In some embodiments, the gene editing systems of the present disclosure can edit and/or target EPSP synthase or any other gene that upon gene editing modifications can produce improved traits.

### Modes and Effects of Gene Editing

The genes of the present disclosure can be edited via various modes resulting in various effects. For example, in some embodiments, the genes can be edited under in vitro culture conditions. In some embodiments, the genes are edited in an environment that does not require gene editing to occur in tissue cultures. In some embodiments, the genes are edited in a greenhouse or similar environment. In some embodiments, the method does not require gene editing to occur through transgenesis.

In some embodiments, the gene edited target cells give rise to gametes. In some embodiments, the gametes can produce gene edited seeds upon fertilization. In some embodiments, homozygote gene edited seeds are produced upon fertilization. In some embodiments, heterozygote gene edited seed are produced upon fertilization.

### APPLICATIONS AND ADVANTAGES

The present disclosure can have various advantages. For instance, in some embodiments, the gene editing methods of the present disclosure utilize tissue-culture independent gene editing of shoot apical meristem (SAM) cells by a long-distance RNA transport system. In some embodiments, the gene editing methods of the present disclosure use natural RNA mobility in plants to supply cells in the SAM with the genetic components required for gene editing without the need of the target cells to be directly subjected to the process of DNA, RNA, or protein delivery.

As such, the gene editing methods of the present disclosure can be utilized in various manners and for various purposes. For instance, in some embodiments, the gene editing methods use natural RNA mobility in plants to supply cells in the SAM with the genetic components required for gene editing without the need of the target cells to be directly subjected to the process of DNA, RNA, or protein delivery or any process of tissue culture or regeneration.

In some embodiments, the gene editing RNAs, Cas9 mRNA, sgRNA(s), and any other required genetic component is produced in, for example, leaves or cotyledons of treated plants and then transported to the SAM. In some embodiments, these genetic components are transcribed, translated and/or processed to have the complete gene editing machinery synthesized in SAM cells that give rise to gametes. To achieve this, the corresponding mRNA are tailored with a long-distance movement signal or zip-code sequence to render the gene editing RNAs mobile and capable of entering into meristematic cells. In SAM cells, Cas9 nuclease and the mature RNA guides are produced to achieve gene editing. Gene-edited cells are still a part of the normal apical meristem, which can later give rise to gametes that, upon fertilization, will produce gene edited seeds.

In some embodiments, transfected leaf or cotyledon cells contain the introduced genetic components required to produce the editing machinery. Thereafter, only the RNA components from the genetic components move into meristematic cells. In some embodiments, this prevents undesirable DNA insertions, genetic and epigenetic alterations in the genome of the target cells due to the Agrobacterium-mediated transformation process or abnormal developmental processes, such as, for example, de novo meristem formation from differentiated cells. Furthermore, mobile RNAs required for gene editing by transient expression in leaves or cotyledons of greenhouse grown plants can also be transported to the SAM where gene editing can take place.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Tissue Culture-Independent Gene Editing of Shoot Apical Meristem Cells by a Long-Distance RNA Transport System

In this Example, Applicant describes a tissue culture-independent gene editing system of shoot apical meristem cells. The gene editing system includes a long-distance RNA transport system as described herein.

In this Example, Applicant targeted the Phytoene Desaturase (PDS) genes of Nicotiana benthamiana. In this model, there are two PDS homolog genes, PDS1 (Niben101Scf01283Ctg022) and PDS2 (Niben101Scf01283g02002.1). PDS genes have been commonly chosen as targets because the mutant phenotype is easy to track. The loss of function of all PDS genes produces a white phenotype due to chlorophyll photobleaching.

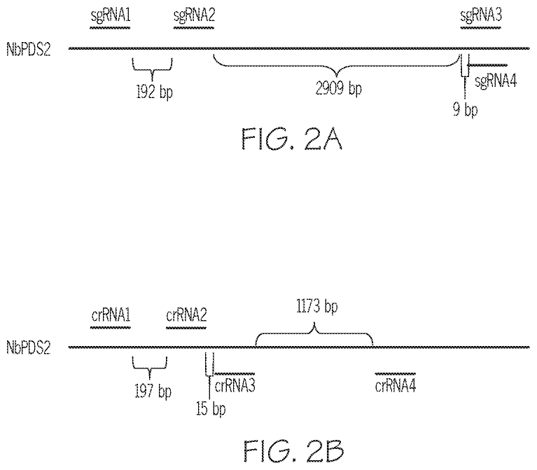

The main feature of Applicant's strategy in this Example was to take advantage of the translocating capabilities of the Arabidopsis thaliana FLOWERING LOCUS T (FT) gene, which has been proven to aid in the translocation of a nearly 6.5 Kbp naked RNA virus. Applicant designed Cas9 (sgRNAs) and CasΦ2 (CasΦ) CRISPR RNAs (crRNAs). To select the guides, Applicant selected conserved regions between both PDS homologs in Nicotiana benthamiana and Nicotiana tabacum plants. Applicant can use the online tool CHOP-CHOP (https://chopchop.cbu.uib.no) or similar systems to design four Cas9 sgRNAs and four CasΦ crRNAs (FIGS. 2A-2B and Table 1). The chosen guide RNAs can be used to target both PDS genes in the two tobacco species.

TABLE 1
Cas9 and CasΦ guide sequences.

| Cas9 | |
|---|---|
| guides | Target sequence |
| sgRNA1 | TTGGTAGTAGCGACTCCATG (SEQ ID NO: 5) |
| sgRNA2 | TACAGTTAACTATTTGGAGG (SEQ ID NO: 6) |
| sgRNA3 | CTCTTGCCAGCAATGCTTGG (SEQ ID NO: 7) |
| sgRNA4 | GATTGCCCTCCAAGCATTGC (SEQ ID NO: 8) |
| CasΦ | |
| guides | |
| crRNA1 | GTAGTAGCGACTCCATGGGG (SEQ ID NO: 9) |
| crRNA2 | ACTATTTGGAGGCGGCGTTA (SEQ ID NO: 10) |
| crRNA3 | GTTGGGCGTGAGGAAGTACG (SEQ ID NO: 11) |
| crRNA4 | CCTCCAGCAATATCGGTTTG (SEQ ID NO: 12) |

Applicant then moved to the design of the editing vectors. Applicant aimed to overexpress the editing reagents and force the plants to produce as many transgene transcripts as possible. Therefore, Applicant integrated the replication machinery of the Bean Yellow Dwarf Virus (BeYDV) in their vectors, a strategy previously proposed (Biotechnology and bioengineering, 2006, 93(2), 271-279).

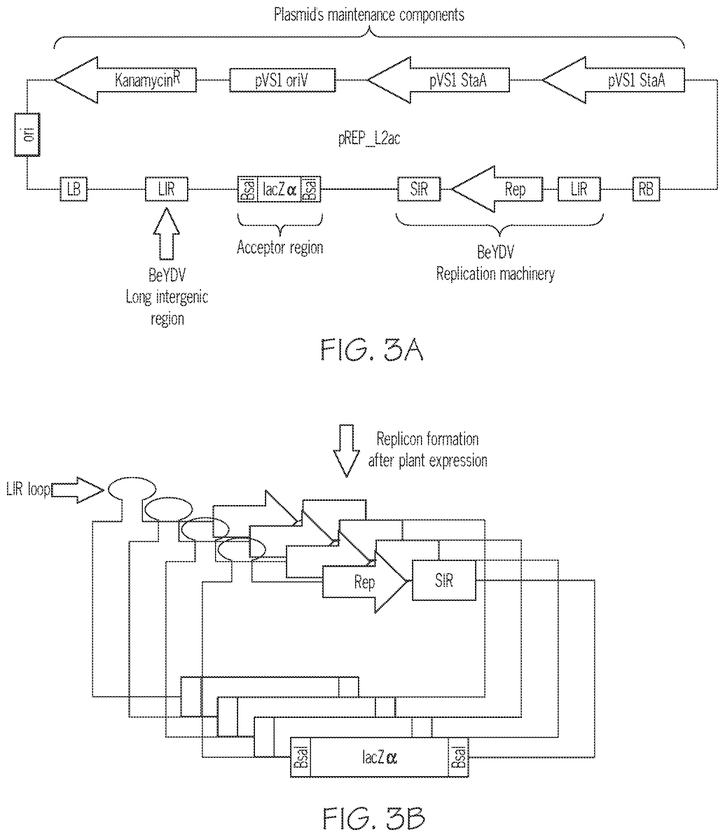

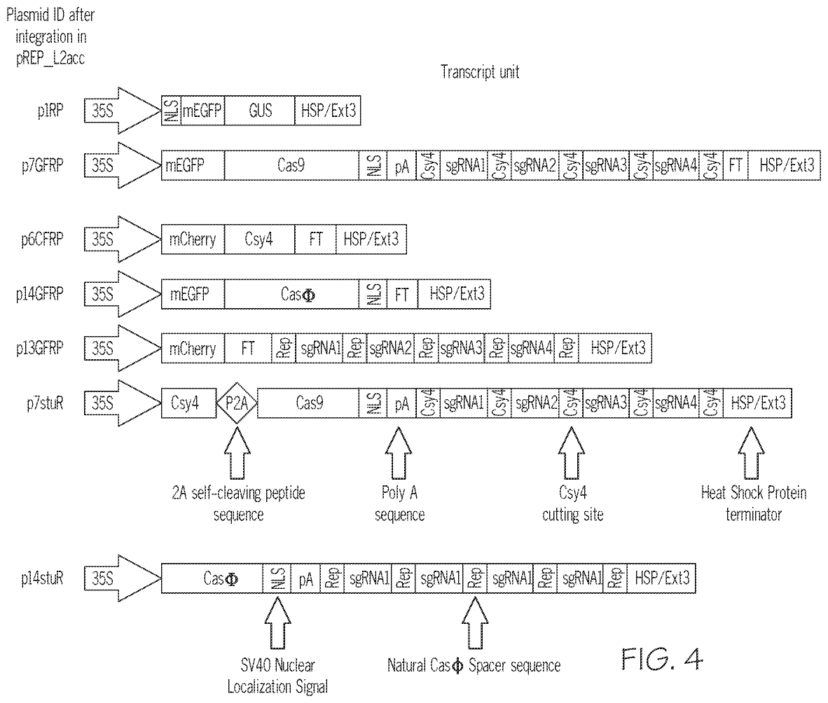

Applicant also aimed to have the flexibility to evaluate different gene constructions. Hence, Applicant constructed a binary Golden Gate Level 2 acceptor vector (FIGS. 3A-3B) by cloning the replication machinery of BeYDV from pREP (Addgene plasmid #51491) into a Golden Gate level 2 acceptor vector from the MoClo Tool Kit plasmid collection (Addgene Kit #1000000044). This acceptor vector was designed to accommodate one complete Golden Gate level 1 transcript unit. Applicant used pREP_L2acc plasmid to assemble the constructions in FIG. 4.

The Csy4 and Csy4: P2A: Cas9 coding sequences were amplified and cloned into Golden Gate Level 0 plasmids from the pDIRECT_22C plasmid (Addgene plasmid #91135). The CasΦ CDS, Heat Shock Protein Terminator, Flowering Locus T gene CDS (FT), and all the sgRNAs and crRNAs tandems were synthesized by the company Gene script and were integrated into Level 0 plasmids. The remaining parts were contained within the MoClo Plant Parts plasmid collection (Addgene Kit #1000000047).

In the CRISPR/Cas systems, the guide RNAs and the Cas nuclease are two separate editing reagents. Applicant designed individual plasmids to produce a single mRNA containing the Cas9 nuclease coding sequence and the four sgRNAs spaced with Csy4 cut sites (FIG. 4, construct p7GFRP). To process the Cas9/sgRNAs, Applicant produced a second plasmid coding for Csy4, the nuclease that naturally processes sgRNAs in bacteria (FIG. 4, construct p6CFRP). These two original transcription units, Cas9/sgRNAs and Csy4, contain the whole FT CDS sequence as part of the 3′ UTR, but lacking an initiation codon to prevent its translation. We included the FT sequence to promote transport of the Cas9/sgRNAs and Csy4 transcript into the meristem of Agro-inoculated plants.

Without being bound by theory, Applicant hypothesized that smaller transcripts would move more efficiently in the vascular system of plants to reach meristematic cells. Hence, Applicant generated constructions containing the recently characterized CasΦ nuclease (Science, 2020, 369(6501), 333-337). CasΦ is half the size of SpCas9 and is a type V CRISPR-Protein that can process its own crRNAs. Therefore, Applicant designed and constructed a gene encoding the CasΦ nuclease linked to an untranslatable FT coding sequence (FIG. 4, construct p14GFRP) and a complementary plasmid with a gene containing the FT sequence linked to the tandem crRNAs to target the PDS genes. Each crRNA is spaced by the natural CasΦ crRNA processing sites. Thus, upon production of CasΦ, the crRNAs are cleaved into active guides RNAS.

In addition to the split editing systems in which the nuclease genes and guide RNAs were produced as separate transcripts, Applicant also produced Single Transcriptional Units for both CRISPR/Cas systems. This approach combines the nuclease sequences, the guide RNAs, and the RNA processing machinery into a single transcriptional unit driven by a single promoter. The translatable part of the transcripts is separated from the guide RNA tandem by a synthetic poly Adenine sequence (50×A). For the Cas9 Single Transcript Unit, the processing Csy4 enzyme is separated from Cas9 by a 2A self-cleaving peptide sequence to separate the two polypeptides after translation (FIG. 4 constructs p7stuR and p14stuR). This sequence can induce ribosomal skipping during translation, splitting the forming peptide and allowing equimolar amounts of the two enzymes (Nature biotechnology, 2004, 22 (5), 589-594).

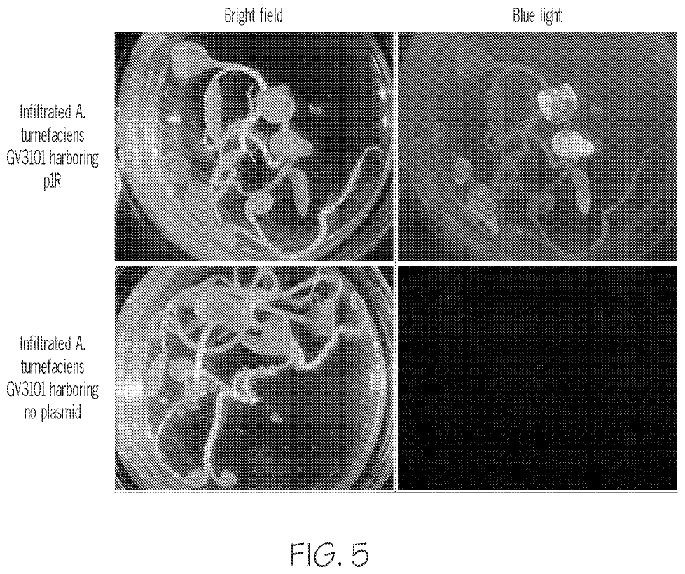

To test the expression of the constructs described in FIG. 4, Applicants used the published Fast-TrACC methodology (Frontiers in genome editing, 2021, 2, 32). In this protocol, one-week-old seedlings are co-cultivated with the bacterial strain in liquid MS:AB media for two days before evaluating transgene expression. Applicant initially used Agrobacterium tumefaciens GV3101 with the p1RP vector to visualize fluorescence (FIG. 5). Overall, transgene expression induced by the Fast-TrACC protocol was variable and required induction of the Agrobacterium Vir genes prior to Agroinfiltration.

Due to the variable results obtained with the original TrACC methodology, Applicant decided to simplify the method and include a vacuum infiltration step to make the system more reproducible. Applicant also evaluated the protocol with the Rhizobium rhizogenes 18r12 strain. In the protocol, N. benthamiana surfaced sterilized seeds are placed in twelve wells plates, with 4-6 seeds per well. Each plate contains 750 μL of 0.5×MS media with 5 g/L Sucrose. The seeds are then left to germinate (around 5 days). Three days after germination, the seedlings are placed in 50 mL conical tubes and vacuum infiltrated at −0.75 bar for 15 minutes with a 0.5×MS media with 5 g/L Sucrose and 200 uM acetosyringone containing the desired Agrobacterium strain at an optic density of 0.14 as measure at a wavelength of 600 nanometers. The seedlings were then rinsed in sterile deionized water once and placed into clean twelve well plates containing 750 uL 0.5×MS media with 5 g/L Sucrose and 200 uM acetosyringone. Three days after cocultivation, seedlings are washed with water containing 200 mg/L Cefotaxime and 0.2% Plant Preservative Media to eliminate the Agrobacterium strain to transfer the target plasmids. After that, seedlings were returned into twelve well plates containing fresh MS media to evaluate GFP fluorescence as a result of expression of target constructs.

For the preparation of the infiltration solution, an initial 5 mL LB media pre-inoculum is prepared in 50 mL conical tubes for each bacterial strain with the following antibiotic concentrations: For Agrobacterium tumefaciens GV3101 50 mg/L Kanamycin, 30 mg/L Gentamicin, and 10 mg/L Rifampicin. For Rhizobium rhizogenes 18r12 50 mg/L Kanamycin and 100 mg/L Streptomycin. Then 100 uL from each strain are cultivated in 10 mL fresh LB media containing only 50 mg/L Kanamycin for all strains and left to grow overnight at 28° C. in an orbital shaker at 225 RPM. Then, the LB media is discarded by centrifuging the cells for 10 minutes at 4000 RPM. The cells are then resuspended in 10 mL fresh 0.5×MS media with 5 g/L Sucrose and 200 uM acetosyringone and incubated for one hour at 28° C. in an orbital shaker at 225 RPM. After that, cell density is adjusted to an OD600 of 0.14 prior to infiltrating the plant seedlings.

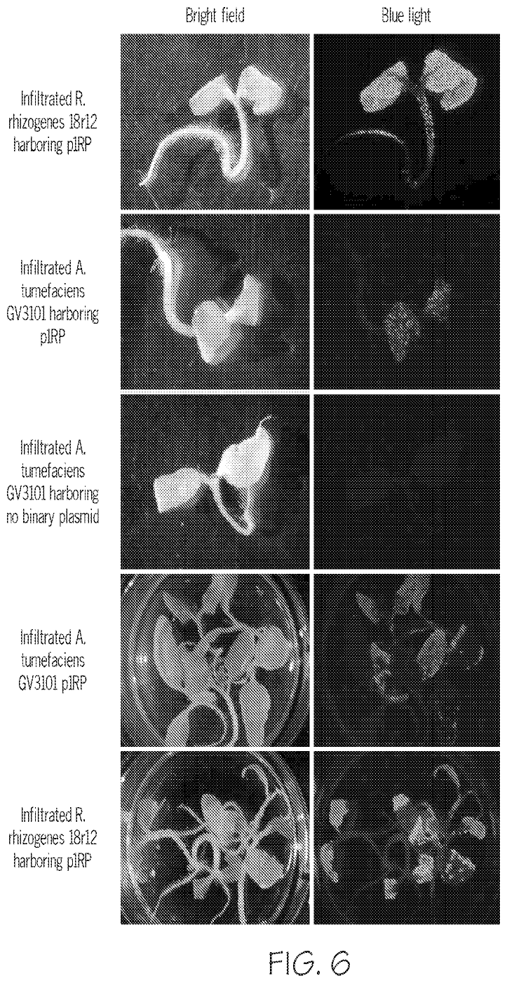

Applicant observed that the aforementioned protocol efficiently produced a transient expression in one-week-old N. benthamiana seedlings. Moreover, Applicant observed that R. rhizogenes performed better than A. tumefaciens in inducing transgene expression (FIG. 6).

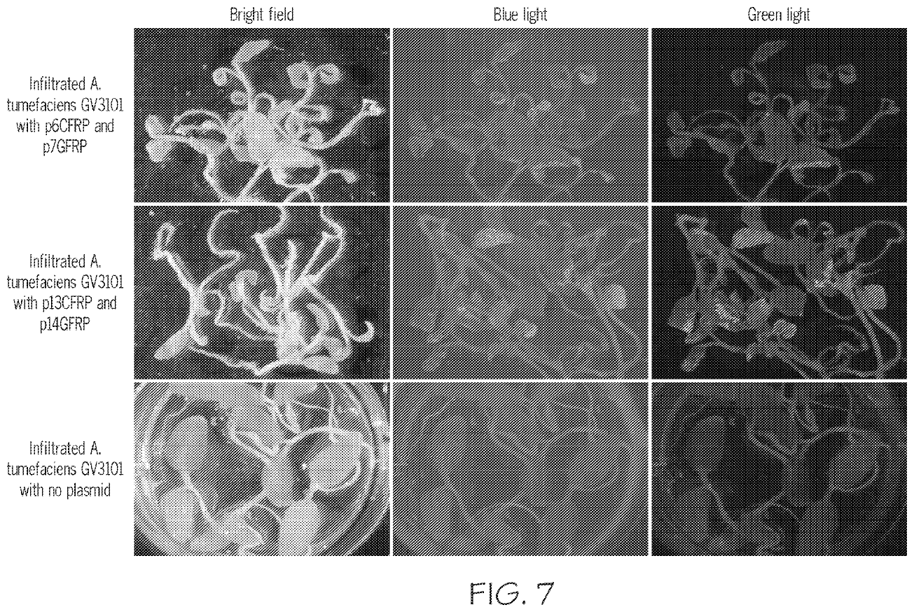

After optimizing the protocol, Applicant infiltrated new seedlings with A. tumefaciens GV3101 harboring Applicant's editing constructions. For instance, Applicant tested the vectors 7stuR and 14stuR individually. Applicant also tested a 1:1 combination of p6CFRP and p7GFRP; and p13CFRP and p14GFRP. Some of Applicant's constructions contained fusions with fluorescent proteins to track the expression of the editing reagents (FIG. 7).

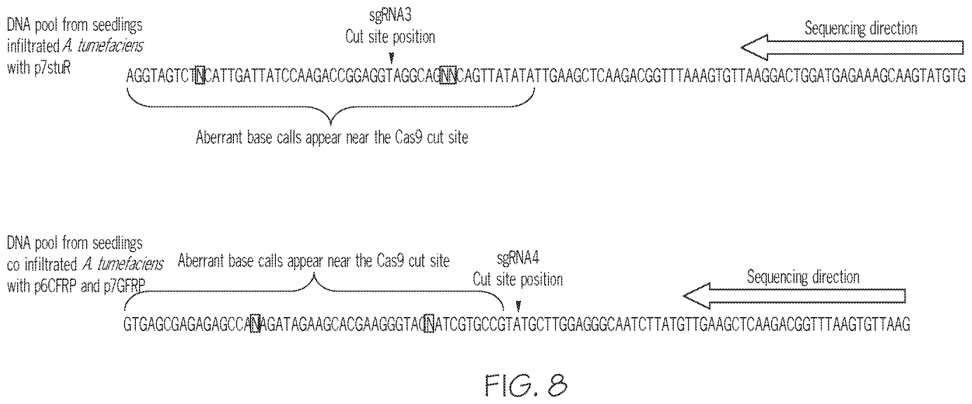

To determine whether the single or split constructs were able to edit the target gene, Applicant pooled seedlings from each treatment and extracted genomic DNA with the CTAB protocol. Applicant then selectively amplified the PDS genes from the DNA extracted from the seedlings Agroinfiltrated with each of the editing constructs and the PCR products subjected to Sanger sequencing (FIG. 8). Interestingly, background noise from aberrant base calls was present in the analyzed electropherograms, showing that gene editing had occurred in the plantlets Agroinfiltrated with the split and single constructs.

Since Applicant used several sgRNAs to target the PDS gene, Applicant will obtain a mixture of small deletions, insertions, base changes, and much larger deletions, making an estimation of the gene editing frequency difficult. Nevertheless, Applicant could observe aberrant base calls near the expected Cas9 cut site (FIG. 8). After an editing event, the position of the wild-type nucleotides is shifted. Polling all the editing events into one sample produced aberrant base calls in Applicant's sequenced samples, suggesting effective gene-editing.

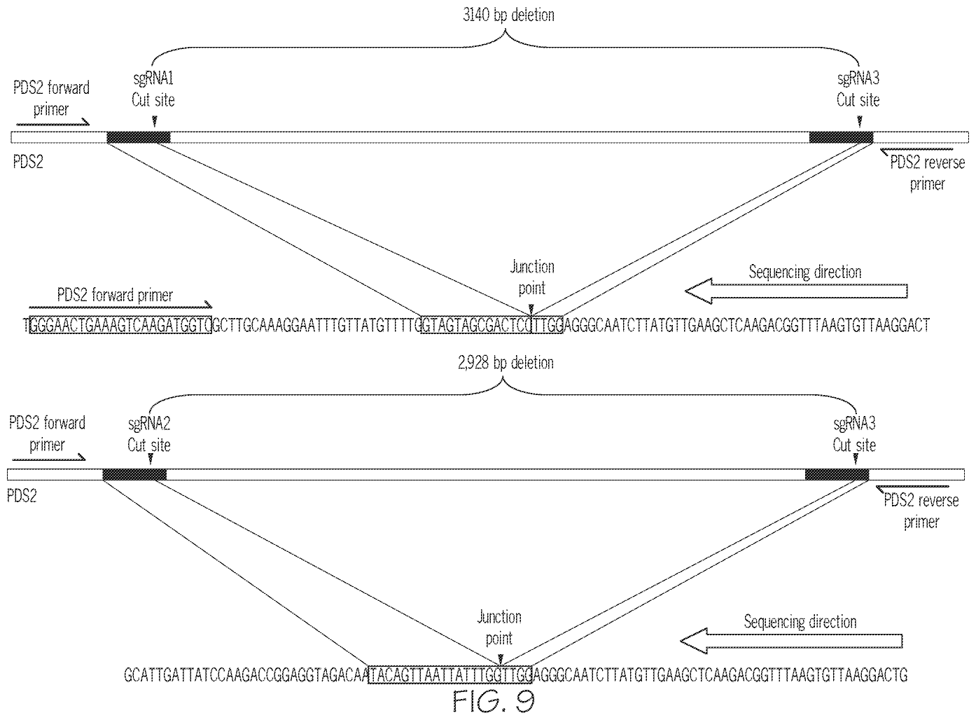

Using short-time amplification PCR to enrich small amplicons allowed Applicants to identify the largest deletions and confirm that the vectors are functional (FIG. 9). So far, Applicant have been able to characterize in detail a 3,140 bp deletion in the pool of plants treated with Agrobacterium tumefaciens GV3101 harboring p7stuR (FIG. 9) and a 2,928 bp deletion in seedlings treated with co-cultivated Agrobacterium tumefaciens GV3101 harboring p6CFRP and p7GFRP (FIG. 9), both fit with the distance of expected from the designed sgRNAs.

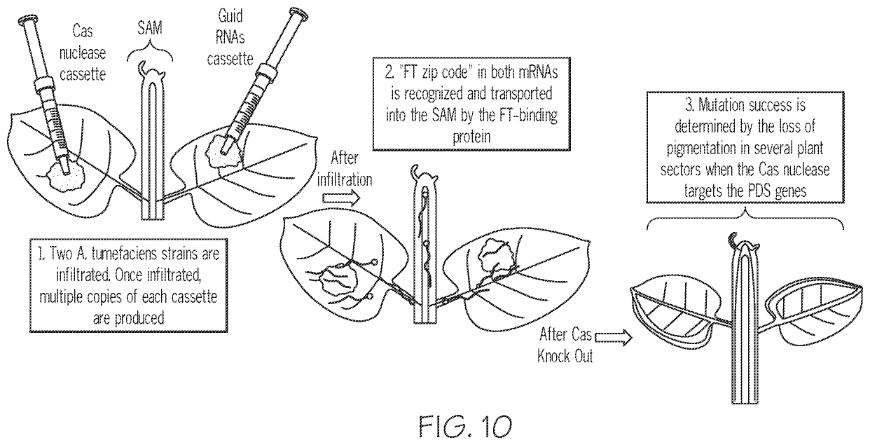

After proving that Applicant's vectors are functional and can indeed produce targeted mutations, Applicant moved forward to evaluate the translocation capabilities of the transgenes. A general representation of Applicant's system is shown in FIG. 10. In principle, any methodology capable of transferring exogenous DNA to be expressed in plant leaves can be used. For instance, the bacteria Rhizobium rhizogenes is a potential option in which Applicant has observed greater transgene expression in seedlings when compared to Agrobacterium tumefaciens. Applicant have also considered T-DNA-free strategies in which non-biological tools are used. Potential alternatives involve using a gene gun to deliver Applicant's mobile editing reagents through particle bombardment. The recently published use of carbon nanotubes is even more promising in which DNA is bound to carbon particles through electrostatic adsorption and infiltrated into plant leaves. That methodology has been proven to work in multiple plant species (Nature Protocols, 2019, 14(10), 2954-2971). The main advantage of Applicant's methodology is that there is no transgene integration at any moment in the meristematic target cells.

As a proof of concept, Applicant used A. tumefaciens GV3101 to induce transgene expression. A. tumefaciens GV3101 electrocompetent cells were electroporated with the plasmids p6CFRP, p7GFRP, and pREP_L2acc vector. The cells were left to recover for 1 hour in SOC media at 28° C. in a thermos saker and plated on LB media (10 g/L Tryptone, 10 g/L NaCl, 5 g/L Yeast Extract, pH 7) with selection antibiotics (50 mg/L Kanamycin, 30 mg/L Gentamicin, and 10 mg/L Rifampicin) and incubated at 28° C. for 2 days. After that, colony PCR was performed to verify the presence of each plasmid. The selected colonies were then incubated in 50 m1 conical tubes with 10 mL liquid LB media with the corresponding antibiotics inside an orbital shaker at 225 RPM for 2 days. After that, the cultures were centrifuged at room temperature for 10 min and 4000 RPM. Next, the supernatant was discarded, and the cells were resuspended to an OD₆₀₀ of 0.6 in MMA media (10 mM MES, 10 mM MgCl2, 200 uM acetosyringone, pH 5.6). The cultures were placed back into the orbital shaker for 1 hour. The cell density was readjusted to 0.6 before the infiltration.

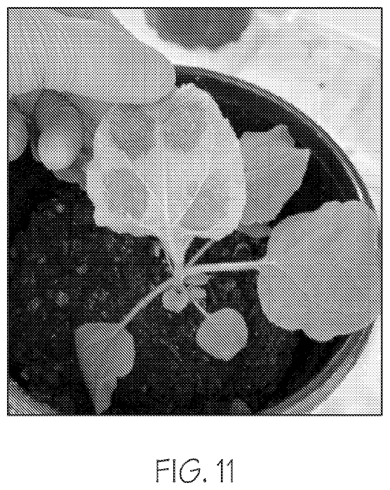

Applicant used 1 mL needleless syringes to infiltrate into two different leaves of 6 weeks-old N. benthamiana plants. Applicant infiltrated 4 times the abaxial surface of the 5^{th} leaf (from bottom to top) with A. tumefaciens GV3101 (FIG. 11), harboring the p6CFRP plasmid, and immediately after, Applicant infiltrated the 6^{th} leaf with A. tumefaciens GV3101 harboring the p7GFRP plasmid. Applicant also infiltrated other plants with bacteria carrying the empty pREP_L2acc vector in the 5^{th} and 6^{th} leaves as a negative control. Four days after the infiltration, Applicant removed the apex of 3 plants infiltrated with the editing reagents and 3 with the PREP_L2acc vector.

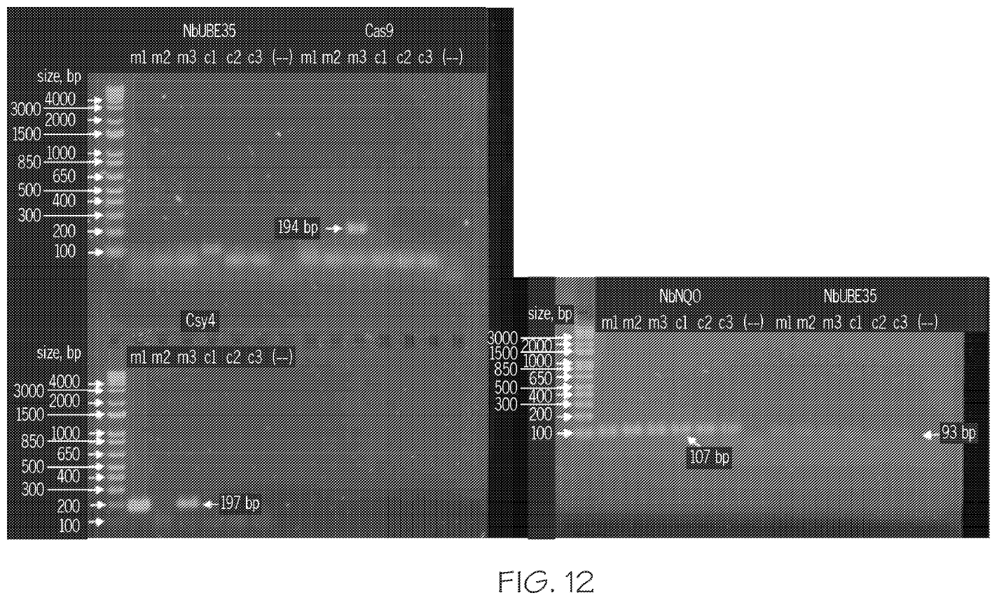

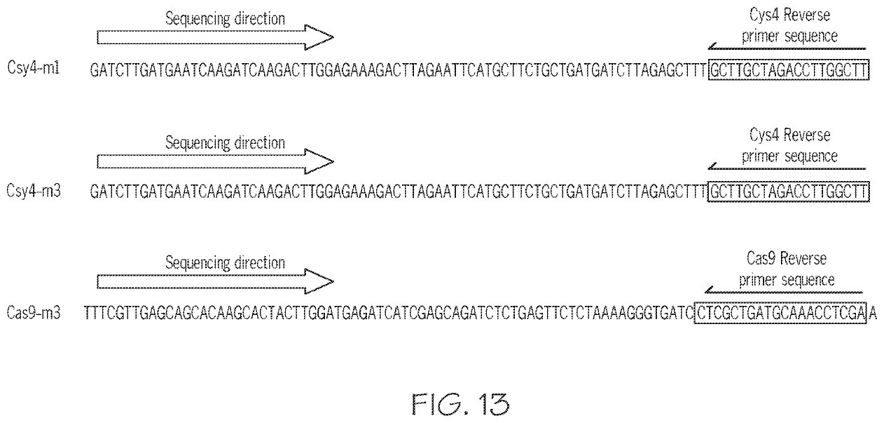

Applicant extracted total RNA and performed an RT-PCR to assess the presence of the Cas9 and Csy4 transcripts. Applicant found that one of the apexes from the plants infiltrated with the editing reagents (m1) contained the Csy4 transcript, and a second apex (m3) contained both Cas9 and Csy4 transcripts (FIG. 12). Applicant subjected the amplicons to Sanger sequencing to verify the identity of the transcripts (FIG. 13) and found that, indeed, they correspond to the Csy4 and Cas9 transcripts. Since the transcripts of both editing reagents are present in the m3 apex, Applicant envisions that the long-distance RNA transport system described in this Example can edit the target genes of meristematic tissues.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of editing at least one gene in plant target cells, said method comprising:
introducing genetic components of a gene editing system to a first region of the plant,
wherein the target cells are located in a second region of the plant that is different from the first region,
wherein the genetic components are transported from the first region to the second region,
wherein the genetic components are processed in the target cells in the second region to form the gene editing system, and
wherein the gene editing system edits the at least one gene in the target cells.

2. The method of claim 1, wherein the plant is selected from the group consisting of maize, rice, soybean, cotton, wheat, N. benthamiana, Arabidopsis, tobacco, tomato, lettuce, common beans, potato, grapes, varieties thereof, and combinations thereof.

3. The method of claim 1,
wherein the first region is selected from the group consisting of leaves, stems, cotyledons, and combinations thereof, and
wherein the second region is selected from the group consisting of shoot apical meristem, floral meristem, inflorescence meristems, root apical meristem, lateral meristems, and combinations thereof.

4-6. (canceled)

7. The method of claim 1, wherein the second region comprises a floral meristem.

8. (canceled)

9. The method of claim 1, wherein the target cells are capable of forming gametes or give rise to gamete forming cells, and wherein the gametes are capable of forming seeds upon fertilization.

10. (canceled)

11. The method of claim 1, wherein the target cells are selected from the group consisting of meristematic cells, shoot apical meristematic cells, floral meristematic cells, inflorescence meristem cells, root apical meristematic cells, lateral meristem cells, and combinations thereof.

12. (canceled)

13. The method of claim 1, wherein the target cells comprise floral meristematic cells.

14. The method of claim 1, wherein the genetic components are introduced in the form of DNA, and wherein the DNA is transcribed into one or more RNAs in the first region and then transported to the second region, wherein the RNA comprises one or more transcripts of the gene editing system and a transport signal for an RNA transport system of the plant, wherein the transport signal is operative for facilitating the transport of the RNA from the first region to the second region of the plant by the RNA transport system.

15-17. (canceled)

18. The method of claim 1, wherein the genetic components are introduced in the form of RNA, and wherein the RNA is transported to the second region.

19. The method of claim 1, wherein the introduction occurs by a method selected from the group consisting of transfection, electroporation, particle bombardment, agrofiltration, and combinations thereof.

20. The method of claim 1, wherein the introduction occurs through a bacterial host strain carrying the genetic components, wherein the bacterial host strain comprises at least one of A. tumefaciens and R. rhizogenes.

21-24. (canceled)

25. The method of claim 1, wherein the editing comprises introducing a mutation to the gene, introducing a deletion to the gene, introducing an insertion to the gene, removing a portion of the gene, changing a base of the gene, removing the gene, inserting the gene, partially or fully replacing the gene, and combinations thereof.

26. (canceled)

27. The method of claim 1, wherein the gene edited cells give rise to gametes that produce gene edited seeds upon fertilization.

28. The method of claim 1, wherein the gene editing system comprises a clustered regularly interspaced short palindromic repeats (CRISPR)/Cas nuclease (Cas) system (CRISPR/Cas system), wherein the CRISPR/Cas system comprises at least one Cas nuclease and at least one guide RNA.

29. The method of claim 28, wherein the genetic components of the CRISPR/Cas system comprise:
the genetic components of the Cas nuclease and a guide RNA precursor,
the genetic component of a guide RNA nuclease, wherein the guide RNA nuclease is operable to convert the guide RNA precursor to the guide RNA, and
at least one transport sequence, wherein the at least one transport sequence is recognizable by an RNA transport system for facilitating the transport of the genetic components from the first region to the second region.

30-35. (canceled)

36. The method of claim 28,
wherein the genetic components of the CRISPR/Cas system are introduced in the form of DNA,
wherein the DNA encodes the at least one guide RNA and the at least one Cas nuclease; wherein the DNA is transcribed into one or more RNAs in the first region, wherein the one or more RNAs are transported to the second region, and wherein the one or more RNAs are processed in the target cells in the second region to form the CRISPR/Cas system; and
wherein the processing comprises translation of the one or more RNAs to form the Cas nuclease, the cutting of the one or more RNAs by the Cas nuclease to form the guide RNA, and the association of the Cas nuclease with the formed guide RNA to form the CRISPR/Cas system.

37. The method of claim 36, wherein the DNA of the genetic components are contained in a single expression vector.

38. The method of claim 36, wherein the DNA of the genetic components are contained in a first and a second expression vector, wherein the first expression vector expresses the Cas nuclease, and wherein the second expression vector expresses the guide RNA.

39. The method of claim 36, wherein the Cas nuclease comprises CasΦ fused to at least one nuclear localization peptide.

40. The method of claim 28,
wherein the genetic components of the CRISPR/Cas system are introduced in the form of DNA,
wherein the DNA encodes the at least one guide RNA, the at least one Cas nuclease, and at least one guide RNA nuclease;
wherein the DNA is transcribed into one or more RNAs in the first region, wherein the one or more RNAs are transported to the second region, and wherein the one or more RNAs are processed in the target cells in the second region to form the CRISPR/Cas system; and
wherein the processing comprises translation of the one or more RNAs to form the Cas nuclease and the guide RNA nuclease, the cutting of the one or more RNAs by the guide RNA nuclease to form the guide RNA, and the association of the Cas nuclease with the formed guide RNA to form the CRISPR/Cas system.

41. The method of claim 40, wherein the DNA of the genetic components are contained in a single expression vector.

42. The method of claim 40, wherein the DNA of the genetic components are contained in a first and a second expression vector, wherein the first expression vector expresses the Cas nuclease and the guide RNA, and wherein the second expression vector expresses the guide RNA nuclease.

43. The method of claim 40, wherein the Cas nuclease comprises Cas 9 fused to at least one nuclear localization peptide, and wherein the guide RNA nuclease comprises Csy 4.

44-49. (canceled)

50. The method of claim 28,
wherein the genetic components of the CRISPR/Cas system are introduced in the form of one or more RNAs;
wherein the one or more RNAs comprise a precursor to the at least one guide RNA, a messenger RNA for the at least one Cas nuclease, and a messenger RNA for at least one guide RNA nuclease;
wherein the one or more RNAs are transported to the second region, and wherein the one or more RNAs are processed in the target cells in the second region to form the CRISPR/Cas system; and
wherein the processing comprises translation of the Cas messenger RNAs to form the Cas nuclease, the translation of the guide RNA nuclease messenger RNA to form the guide RNA nuclease, the cutting of the precursor to the at least on guide RNA by the guide RNA nuclease to form the guide RNA, and the association of the Cas nuclease with the formed guide RNA to form the CRISPR/Cas system.

51. (canceled)
